(19) United States
(12) Patent Application Publication
(10) Pub. No.: US 2024/0308961 A1
(43) Pub. Date: Sep. 19, 2024

(54) Purification Method For 3-Amino-5-Methylpiperidine With Amino Protected By Boc

(71) Applicants: ZHEJIANG MEDICINE CO., LTD. XINCHANG PHARMACEUTICAL FACTORY, Shaoxing (CN); ZHEJIANG CHANGHAI PHARMACEUTICALS CO., LTD., Shaoxing (CN)

(72) Inventors: Li SHENG, Shaoxing (CN); Xufeng WU, Shaoxing (CN); Gang FAN, Shaoxing (CN); Yuquan LUO, Shaoxing (CN); Lanfang ZHU, Shaoxing (CN); Long CHEN, Shaoxing (CN); Ruiwei CAO, Shaoxing (CN); Dadong SHEN, Shaoxing (CN)

(21) Appl. No.: 18/510,699

(22) Filed: Nov. 16, 2023

(30) Foreign Application Priority Data
Mar. 15, 2023 (CN) 202310276043.1

(51) Int. Cl. C07D 211/56

(52) U.S. Cl. CPC C07D 211/56

(57) ABSTRACT

The present disclosure provides a purification method for 3-amino-5-methylpiperidine with amino protected by Boc. The method includes adding a solvent to the crude product of 3-amino-5-methylpiperidine with amino protected by Boc, heating until it is dissolved to obtain a mixed solution; adding an acid and an amine crystallization auxiliary to the mixed solution, stirring and dispersing to obtain a dispersed solution; cooling the dispersed solution until crystallization, separating and drying same to obtain a salt crystal of 3-amino-5-methylpiperidine with amino protected by Boc. The present disclosure improves the product characters of 3-amino-5-methylpiperidine with amino protected by Boc after salifying and crystallization, thereby improving the yield and purity of the product, at the same time, it simplifies the operation steps of salifying and crystallization of 3-amino-5-methylpiperidine with amino protected by Boc, shortens the operation time, and reduces production costs.

### TECHNICAL FIELD

The present disclosure relates to the technical field of organic synthesis, in particular to a purification method for 3-amino-5-methylpiperidine with amino protected by Boc.

### BACKGROUND

3-amino-5-methylpiperidine is an intermediate in drug synthesis, for example, as an intermediate to synthesize the side chain of a quinolone antibiotic (3S,5S)-7-[3-amino-5-methyl-piperidine]-1-cyclopropyl-1,4-dihydro-8-methoxy-4-oxo-3-quinoline carboxylic acid. Due to the significant toxic side effects that may arise from the related substances and optical isomeric impurities in the drug, it is necessary to strictly control the level of related impurities in the starting material 3-amino-5-methylpiperidine.

At present, 3-amino-5-methylpiperidine is generally synthesized from natural D-glutamic acid with amino protected by Boc as raw material through multi-step reactions to obtain 3-amino-5-methylpiperidine with amino protected by Boc (as shown below).

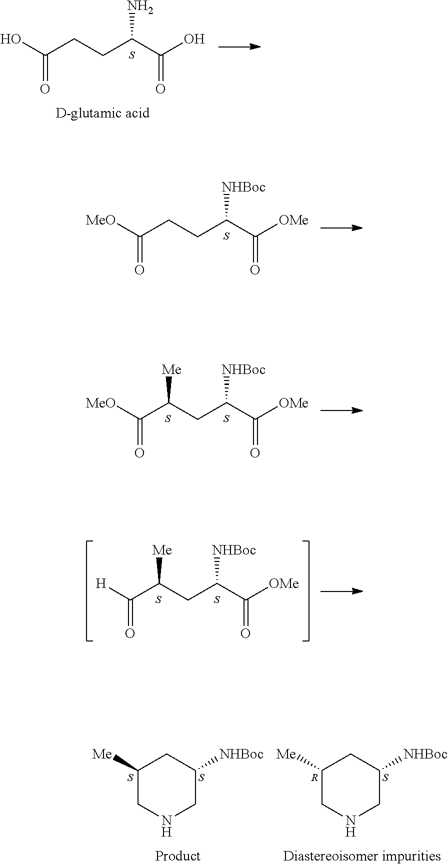

Since D-glutamic acid already contains high purity of chiral amino groups, the main optical impurity in 3-amino-5-methylpiperidine is its diastereoisomer, the removal of the impurity becomes the key point to product quality control. A synthesis method of 3-amino-5-methylpiperidine with amino protected by Boc had been reported in patent US20070232650A1, but there was no purification operation in the post-treatment, resulting in a waxy solid product and failure to obtain high-purity crystals. In Patent US20100152452A1, oxalic acid was added to the reaction system to make the intermediate form an oxalate, but there was no further post-treatment operation to remove diastereoisomer impurities in the product. When common crystallization methods are used, it can be observed that the crystallization rate is slow, and the products before crystallization appear in a colloidal form, thus it is necessary to strictly control the cooling program and crystallization time of crystallization, otherwise some products that have not been converted into crystals can adsorb on the precipitated solid, leading to a higher level of impurities in the product.

### SUMMARY

The main objective of the present disclosure is to provide a purification method for 3-amino-5-methylpiperidine with amino protected by Boc, so as to solve the problems of difficult crystallization and low purity of 3-amino-5-methylpiperidine with amino protected by Boc in the existing technology.

In order to achieve the above object, according to an aspect of the present disclosure, a purification method for 3-amino-5-methylpiperidine with amino protected by Boc as shown in formula I is provided, which comprises the following steps:

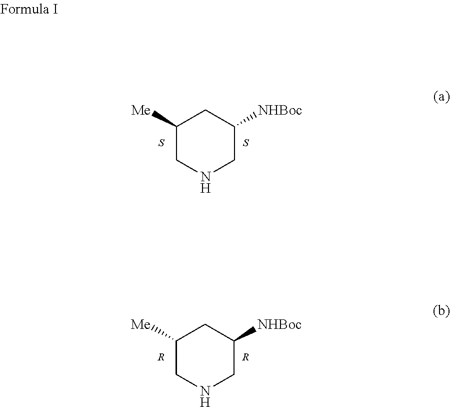

step S1, adding a solvent to the crude product of 3-amino-5-methylpiperidine with amino protected by Boc, heating until it is dissolved to obtain a mixed solution, the crude product comprises (3S,5S) configuration product as shown in formula I (a) and/or (3R,5R) configuration product as shown in formula I (b); step S2, adding an acid and an amine crystallization auxiliary to the mixed solution, stirring and dispersing to obtain a dispersed solution; and step S3, cooling the dispersed solution until crystallization, separating and drying same to obtain a salt crystal of 3-amino-5-methylpiperidine with amino protected by Boc.

Further, in the step S1, the solvent is one or more of methanol, ethanol, n-propanol, and isopropanol.

Further, in the step S1, the liquid-solid ratio of the solvent to the crude product of 3-amino-5-methylpiperidine with amino protected by Boc is (4-16): 1.

Further, in the step S1, the heating temperature is the reflux temperature of the solvent, preferably, the heating temperature is 40° C. to 100° C.

Further, in the step S2, the acid is an organic acid, preferably one or more of formic acid, acetic acid, methanesulfonic acid, p-toluenesulfonic acid, oxalic acid, succinic acid, lactic acid, and tartaric acid, and preferably, the acid is one or more of oxalic acid, lactic acid, and tartaric acid.

Further, in the step S2, 0.001 mol to 0.01 mol of the acid is added to each gram of the crude product of 3-amino-5-methylpiperidine with amino protected by Boc in the mixed solution.

Further, in the step S2, the amine crystallization auxiliary is one or more of ammonia, ethylamine, ethylenediamine, triethylamine, benzylamine, aniline, monoethanolamine, and dimethylformamide, and preferably, the amine crystallization auxiliary is benzylamine and/or aniline.

Further, in the step S2, the mass ratio of the amine crystallization auxiliary to the crude product of 3-amino-5-methylpiperidine with amino protected by Boc in the mixed solution is (0.0001-0.1): 1, and preferably, the mass ratio of the amine crystallization auxiliary to the crude product of 3-amino-5-methylpiperidine with amino protected by Boc in the mixed solution is (0.001-0.01): 1.

Further, in the step S2, the dispersion temperature is the reflux temperature of the solvent, and preferably, the dispersion temperature is 40° C. to 100° C.

Further, in the step S3, the crystallization temperature is −20° C. to 30° C., and preferably, the crystallization temperature is 0° C. to 10° C.

Compared with existing technology, the progressiveness of the present disclosure is at least reflected in the following aspects:
1) The present disclosure improves the product characters of 3-amino-5-methylpiperidine with amino protected by Boc after salifying and crystallization, thereby improving the yield and purity of the product, wherein the purity of 3-amino-5-methylpiperidine with amino protected by Boc in the product is a 99.5%, and the content of diastereoisomer impurities is ≤0.15%
(2) The present disclosure simplifies the operation steps of salifying and crystallization of 3-amino-5-methylpiperidine with amino protected by Boc, shortens the operation time, further improving product purity and reducing production costs.

### BRIEF DESCRIPTION OF THE DRAWINGS

The accompanying drawings of the description, which form a part of the present disclosure, are used to provide a further understanding of the present disclosure. The illustrative examples of the present disclosure and their descriptions are used to explain the present disclosure, and do not constitute an improper limitation thereto. In the accompanying drawings:

FIG. 1 shows the liquid phase chromatogram of the related substances of the crude product of 3-amino-5-methylpiperidine with amino protected by Boc according to an example of the present disclosure;

FIG. 2 shows the liquid phase chromatogram of the related substances of the purified product according to example 1 of the present disclosure;

FIG. 3 shows the liquid phase chromatogram of the related substances of the purified product according to comparative example 1 of the present disclosure; and

FIG. 4 shows the liquid phase chromatogram of the related substances of the purified product according to comparative example 2 of the present disclosure.

### DETAILED DESCRIPTION OF THE EMBODIMENTS

It should be noted that the examples in this application and features in the examples may be combined with each other without conflict. The present disclosure will be described in detail below with reference to the accompanying drawings and in combination with examples.

It should be noted that the crude product of 3-amino-5-methylpiperidine with amino protected by Boc in the present disclosure is chemically synthesized from D-glutamic acid or L-glutamic acid with amino protected by Boc as raw material, with reference to patent US20070232650A1 or other feasible synthetic routes, so that 3-amino-5-methylpiperidine is obtained.

### Explanation of Terms

Term “Boc” refers to tert-butoxycarbonyl, i.e. CH₃C(CH₃)₂OCO—*, where “*” refers to the connecting bond.

Term “liquid-solid ratio” refers to the ratio of volume of solution (ml) to solid material (g).

As stated in the background of the present disclosure, there are problems of difficult crystallization and low purity of 3-amino-5-methylpiperidine with amino protected by Boc in the existing technology. It should be noted that “purity” here mainly refers to the purity of 3-amino-5-methylpiperidine with amino protected by Boc having (3S,5S) configuration (referred to as SS configuration) and/or (3R,5R) configuration (referred to as RR configuration) in the crystallization product.

In order to solve the above problems, in a typical embodiment of the present disclosure, a purification method for 3-amino-5-methylpiperidine with amino protected by Boc as shown in formula I is provided, which comprises the following steps:

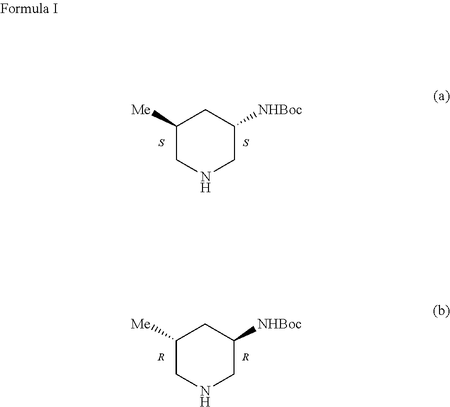

step S1, adding a solvent to the crude product of 3-amino-5-methylpiperidine with amino protected by Boc, heating until it is dissolved to obtain a mixed solution, the crude product comprises (3S,5S) configuration product as shown in formula I (a) and/or (3R,5R) configuration product as shown in formula I (b), and a small amount of diastereoisomer impurities having (3S,5R) configuration and (3R,5S) configuration; step S2, adding an acid and an amine crystallization auxiliary to the mixed solution, stirring and dispersing to obtain a dispersed solution; and step S3, cooling the dispersed solution until crystallization, separating and drying same to obtain a salt crystal of 3-amino-5-methylpiperidine with amino protected by Boc.

According to the present disclosure, a solvent is first added to the Boc-protected 3-amino-5-methylpiperidine, which is heated to reflux until it is dissolved to obtain a mixed solution, and then an acid is added to achieve preliminary purification. During this process, an amine crystallization auxiliary is added, which is then stirred and dispersed to obtain a dispersed solution. If the amine crystallization auxiliary is not added, the precipitated solid product appears in a colloidal form, indicating that the molecular arrangement at the micro-level is disordered, however after the amine crystallization auxiliary is added, the molecular arrangement is reorganized, and crystals are precipitated, so that further purification is realized. Finally, the crystal is separated and dried to obtain high-purity Boc-protected 3-amino-5-methylpiperidine crystals with high yield According to the different configuration characteristics of the reaction raw materials D-glutamic acid or L-glutamic acid, when the reaction raw materials are in monochirality, the final product is SS configuration 3-amino-5-methylpiperidine crystal comprising lower level of other configuration impurities, or RR configuration 3-amino-5-methylpiperidine crystal comprising lower level of other configuration impurities.

The present disclosure improves the product characters of 3-amino-5-methylpiperidine with amino protected by Boc after salifying and crystallization, thereby improving the yield and purity of the product, wherein the purity of 3-amino-5-methylpiperidine with amino protected by Boc in the product is ≥99.5%, and the content of diastereoisomer is ≤0.15%. At the same time, the crystallization operation of salt of 3-amino-5-methylpiperidine with amino protected by Boc is simplified, the operation time is shortened, and the production costs are reduced.

There are no special requirements for the solvent used for dissolving the crude product in the present disclosure, as long as it can form a good dissolution of the crude product and does not affect the subsequent reaction after adding an acid and an amine crystallization auxiliary. In a preferred embodiment, in the step S1, the solvent is one or more of methanol, ethanol, n-propanol, and isopropanol. For the purpose of making the solvent used more compatible with the purification system of the present disclosure, isopropanol is preferred.

In a preferred embodiment, in th step S1, the liquid-solid ratio of the solvent to the crude product of 3-amino-5-methylpiperidine with amino protected by Boc is (4-16): 1, which allows for more suitable dissolution of the crude product and provides a more suitable liquid environment, which is ready for good purification of subsequent products.

Typically but not limited, the liquid-solid ratio of the solvent to the crude product of 3-amino-5-methylpiperidine with amino protected by Boc is 4:1, 5:1, 6:1, 7:1, 8:1, 9:1, 10:1, 11:1, 12:1, 13:1, 14:1, 15:1, 16:1 or a range value consisting of any two of the foregoing ratios.

The heating and dissolution temperature after adding a solvent should not be too low, otherwise it will cause the product to precipitate colloidal solids in advance during subsequent operations, affecting the impurity removal effect, when the temperature is too high, the effect on improving the yield and purity of the product is relatively small, and it can also lead to an increase in cost. Therefore, in a preferred embodiment, in the step S1, the heating temperature is the reflux temperature of the selected solvent, and for the purpose of further improving yield and purity, the heating temperature is preferably 40° C. to 100° C.

In order to further ensure that the reaction between acid and 3-amino-5-methylpiperidine during the salifying process proceeds smoothly, and to achieve both high yield and purity, in a preferred embodiment, in the step S2, the acid is an organic acid, preferably one or more of formic acid, acetic acid, methanesulfonic acid, p-toluenesulfonic acid, oxalic acid, succinic acid, lactic acid, and tartaric acid, and preferably, the acid is one or more of oxalic acid, lactic acid, and tartaric acid, and further preferably oxalic acid. When the above acids are used for salifying reaction, the purity of the product is higher, and the detectable amount of diastereoisomer impurities can be less than 0.1%.

In a preferred embodiment, in the step S2, 0.001 mol to 0.01 mol of the acid is added to each gram of the crude product of 3-amino-5-methylpiperidine with amino protected by Boc in the mixed solution, so that the salifying effect can be further improved and the yield and purity of the product can be increased.

Typically but not limited, the amount of the acid added to each gram of the crude product of 3-amino-5-methylpiperidine with amino protected by Boc in the mixed solution is 0.001 mol, 0.002 mol, 0.003 mol, 0.004 mol, 0.005 mol, 0.006 mol, 0.007 mol, 0.008 mol, 0.009 mol, 0.01 mol, or a range value consisting of any two of the foregoing numerical values.

As mentioned previously, in the existing technologies, the crystallization rate is relatively slow, and it is necessary to strictly control the cooling program and crystallization time of crystallization. Unlike the existing technologies, the use of the amine crystallization auxiliary in the present disclosure can improve the crystallization characters, shorten the crystallization time, and avoid the adsorption of some products that have not been converted into crystals on the precipitated solid due to excessive crystallization time, leading to a higher level of impurities in the product. In a preferred embodiment, in the step S2, the amine crystallization auxiliary is one or more of ammonia, ethylamine, ethylenediamine, triethylamine, benzylamine, aniline, monoethanolamine, and dimethylformamide, which can obtain a crystalline piperidine salt and shorten the crystallization time, preferably the amine crystallization auxiliary includes benzylamine and/or aniline, and further preferably benzylamine. The above amine crystallization auxiliaries can further improve the yield and purity, and the detectable amount of diastereoisomer impurities can be less than 0.1%.

When the addition amount of amine crystallization auxiliary is too low, the purity of the product will be reduced and the detectable amount of diastereoisomer impurities will exceed standards due to the incomplete crystallization of some products and thereby still presenting a colloidal form or adsorbing on the precipitated crystals. When the addition amount is too high, the excessive amine will consume a corresponding amount of acid, resulting in insufficient salifying of the piperidine product and thereby a decreased yield. Therefore, in a preferred embodiment, in the step S2, the mass ratio of the amine crystallization auxiliary to the crude product of 3-amino-5-methylpiperidine with amino protected by Boc in the mixed solution is (0.0001-0.1): 1, and preferably the mass ratio of the amine crystallization auxiliary to the crude product of 3-amino-5-methylpiperidine with amino protected by Boc in the mixed solution is (0.001-0.01): 1. The above mass ratio includes but is not limited to the above ranges, but limiting it to the above ranges is beneficial for further improving the yield and purity of the product.

Typically but not limited, the mass ratio of the amine crystallization auxiliary to the crude product of 3-amino-5-methylpiperidine with amino protected by Boc in the mixed solution is 0.0001:1, 0.0005:1, 0.001:1, 0.005:1, 0.01:1, 0.05:1, 0.1:1 or a range value consisting of any two of the foregoing ratios.

In the present disclosure, an acid and an amine crystallization auxiliary are added to the mixed solution, which is then subjected to insulation stirring to obtain a dispersed solution, and thus it is only needed to keep the dispersion temperature and the heating dissolution temperature consistent. Therefore, in a preferred embodiment, in the step S2, the dispersion temperature is the reflux temperature of the selected solvent, and preferably the dispersion temperature is 40° C. to 100° C.

If the crystallization temperature is too low, the purity of the product will be reduced, and the detectable amount of diastereoisomer impurities will increase. If the crystallization temperature is too high, the yield will be reduced due to incomplete crystallization. Therefore, in a preferred embodiment, in the step S3, the crystallization temperature is −20° C. to 30° C., preferably the crystallization temperature is 0° C. to 10° C., which can further reduce the detectable amount of diastereoisomer impurities and improve the purity of the product.

Typically but not limited, the crystallization temperature is −20° C., −10° C., 0° C., 10° C., 20° C., 30° C., or a range value consisting of any two of the foregoing numerical values.

The present disclosure is further described in detail below in combination with specific examples, and the examples shall not be construed as limitations of the protection scope as required the present disclosure.

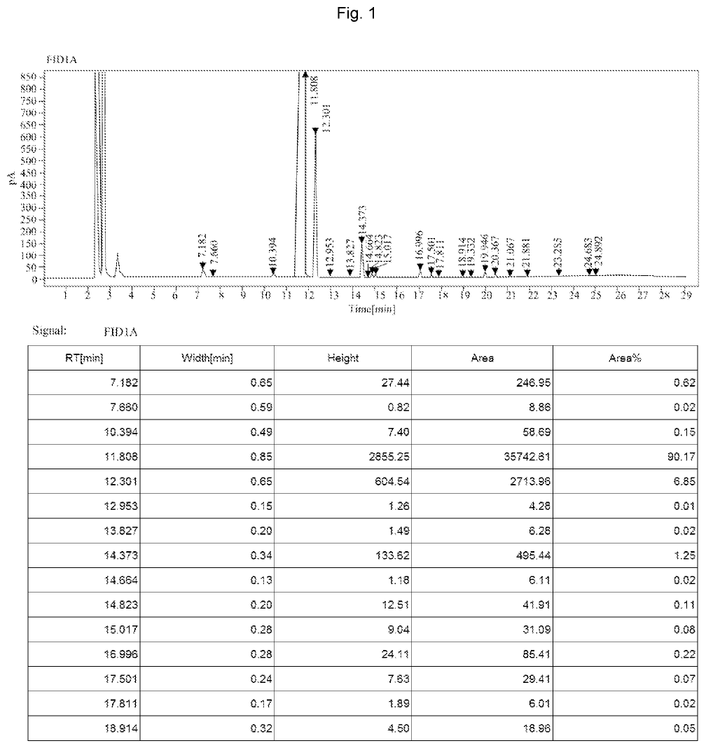

Unless otherwise specified, the crude product of 3-amino-5-methylpiperidine with amino protected by Boc used in the following examples and comparative examples is chemically synthesized from D-glutamic acid or L-glutamic acid with amino protected by Boc as raw material, with reference to patent US20070232650A1 or other feasible synthetic routes, so as to obtain 3-amino-5-methylpiperidine with amino protected by Boc. Upon detection, the principal component 3-amino-5-methylpiperidine with amino protected by Boc contains a content of 90.2% of SS configuration and/or RR configuration, a content of 6.9% of diastereoisomer, and is of moderate alkalinity. The liquid phase chromatogram of the related substances in the crude product is shown in FIG. 1.

Unless otherwise specified, the liquid phase purity and the detectable amount of diastereoisomer in the following examples and comparative examples refer to the mass percentage content.

### Example 1

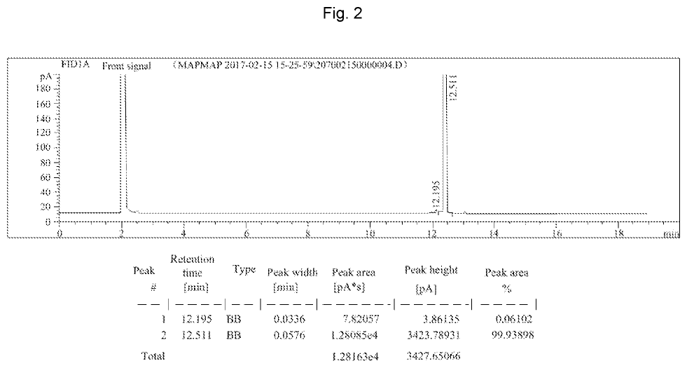

10 g of crude product was taken and 100 ml of isopropanol was added, the mixture was heated to reflux at 90° C., 4.2 g of oxalic acid and 0.05 g of benzylamine were added, and insulation stirred for 30 min, then cooled in an ice water bath to 5° C. for crystallization, which was filtered, the filter cake was dried to obtain a white solid with a yield of 86%, a liquid phase purity of 99.9%, and no diastereoisomer was detected. The liquid phase chromatogram of the related substances in the product is shown in FIG. 2.

### Example 2

10 g of crude product was taken and 100 ml of methanol was added, the mixture was heated to reflux at 40° C., 4.2 g of oxalic acid and 0.05 g of benzylamine were added, and insulation stirred for 30 min, then cooled in an ice water bath to 5° C. for crystallization, which was filtered, the filter cake was dried to obtain a white solid with a yield of 53%, a liquid phase purity of 99.9%, and no diastereoisomer was detected.

### Example 3

10 g of crude product was taken and 100 ml of ethanol was added, the mixture was heated to reflux at 75° C., 4.2 g of oxalic acid and 0.05 g of benzylamine were added, and insulation stirred for 30 min, then cooled in an ice water bath to 5° C. for crystallization, which was filtered, the filter cake was dried to obtain a white solid with a yield of 78%, a liquid phase purity of 99.9%, and no diastereoisomer was detected.

### Example 4

10 g of crude product was taken and 100 ml of n-propanol was added, the mixture was heated to reflux at 100° C., 4.2 g of oxalic acid and 0.05 g of benzylamine were added, and insulation stirred for 30 min, then cooled in an ice water bath to 5° C. for crystallization, which was filtered, the filter cake was dried to obtain a white solid with a yield of 83%, a liquid phase purity of 99.9%, and no diastereoisomer was detected.

### Example 5

10 g of crude product was taken and 40 ml of isopropanol was added, the mixture was heated to reflux at 90° C., 4.2 g of oxalic acid and 0.05 g of benzylamine were added, and insulation stirred for 30 min, then cooled in an ice water bath to 5° C. for crystallization, which was filtered, the filter cake was dried to obtain a white solid with a yield of 89%, a liquid phase purity of 99.5%, and diastereoisomer of 0.14%.

### Example 6

10 g of crude product was taken and 160 ml of isopropanol was added, the mixture was heated to reflux at 90° C., 4.2 g of oxalic acid and 0.05 g of benzylamine were added, and insulation stirred for 30 min, then cooled in an ice water bath to 5° C. for crystallization, which was filtered, the filter cake was dried to obtain a white solid with a yield of 73%, a liquid phase purity of 99.9%, and diastereoisomer of 0.01%.

### Example 7

10 g of crude product was taken and 100 ml of isopropanol was added, the mixture was heated to reflux at 90° C., 4.2 g of lactic acid and 0.05 g of benzylamine were added, and insulation stirred for 30 min, then cooled in an ice water bath to 5° C. for crystallization, which was filtered, the filter cake was dried to obtain a white solid with a yield of 80%, a liquid phase purity of 99.8%, and diastereoisomer of 0.04%.

### Example 8

10 g of crude product was taken and 100 ml of isopropanol was added, the mixture was heated to reflux at 90° C., 7.0 g of tartaric acid and 0.05 g of benzylamine were added, and insulation stirred for 30 min, then cooled in an ice water bath to 5° C. for crystallization, which was filtered, the filter cake was dried to obtain a white solid with a yield of 82%, a liquid phase purity of 99.8%, and diastereoisomer of 0.03%.

### Example 9

10 g of crude product was taken and 100 ml of isopropanol was added, the mixture was heated to reflux at 90° C., 2.2 g of formic acid and 0.05 g of benzylamine were added, and insulation stirred for 30 min, then cooled in an ice water bath to 5° C. for crystallization, which was filtered, the filter cake was dried to obtain a white solid with a yield of 82%, a liquid phase purity of 99.5%, and diastereoisomer of 0.12%.

### Example 10

10 g of crude product was taken and 100 ml of isopropanol was added, the mixture was heated to reflux at 90° C., 2.8 g of acetic acid and 0.05 g of benzylamine were added, and insulation stirred for 30 min, then cooled in an ice water bath to 5° C. for crystallization, which was filtered, the filter cake was dried to obtain a white solid with a yield of 79%, a liquid phase purity of 99.6%, and diastereoisomer of 0.15%.

### Example 11

10 g of crude product was taken and 100 ml of isopropanol was added, the mixture was heated to reflux at 90° C., 4.5 g of methanesulfonic acid and 0.05 g of benzylamine were added, and insulation stirred for 30 min, then cooled in an ice water bath to 5° C. for crystallization, which was filtered, the filter cake was dried to obtain a white solid with a yield of 82%, a liquid phase purity of 99.8%, and diastereoisomer of 0.10%.

### Example 12

10 g of crude product was taken and 100 ml of isopropanol was added, the mixture was heated to reflux at 90° C. 8.0 g of p-toluenesulfonic acid and 0.05 g of benzylamine were added, and insulation stirred for 30 min, then cooled in an ice water bath to 5° C. for crystallization, which was filtered, the filter cake was dried to obtain a white solid with a yield of 80%, a liquid phase purity of 99.8%, and diastereoisomer of 0.11%.

### Example 13

10 g of crude product was taken and 100 ml of isopropanol was added, the mixture was heated to reflux at 90° C., 5.5 g of succinic acid and 0.05 g of benzylamine were added, and insulation stirred for 30 min, then cooled in an ice water bath to 5° C. for crystallization, which was filtered, the filter cake was dried to obtain a white solid with a yield of 83%, a liquid phase purity of 99.7%, and diastereoisomer of 0.11%.

### Example 14

10 g of crude product was taken and 100 ml of isopropanol was added, the mixture was heated to reflux at 90° C., 1 g of oxalic acid and 0.05 g of benzylamine were added, and insulation stirred for 30 min, then cooled in an ice water bath to 5° C. for crystallization, which was filtered, the filter cake was dried to obtain a white solid with a yield of 51%, a liquid phase purity of 99.8%, and diastereoisomer of 0.03%.

### Example 15

10 g of crude product was taken and 100 ml of isopropanol was added, the mixture was heated to reflux at 90° C., 10 g of oxalic acid and 0.05 g of benzylamine were added, and insulation stirred for 30 min, then cooled in an ice water bath to 5° C. for crystallization, which was filtered, the filter cake was dried to obtain a white solid with a yield of 71%, a liquid phase purity of 99.7%, and diastereoisomer of 0.10%.

### Example 16

10 g of crude product was taken and 100 ml of isopropanol was added, the mixture was heated to reflux at 90° C., 4.2 g of oxalic acid and 0.05 g of aniline were added, and insulation stirred for 30 min, then cooled in an ice water bath to 5° C. for crystallization, which was filtered, the filter cake was dried to obtain a white solid with a yield of 84%, a liquid phase purity of 99.8%, and diastereoisomer of 0.04%.

### Example 17

10 g of crude product was taken and 100 ml of isopropanol was added, the mixture was heated to reflux at 90° C., 4.2 g of oxalic acid and 0.05 g of ammonia were added, and insulation stirred for 30 min, then cooled in an ice water bath to 5° C. for crystallization, which was filtered, the filter cake was dried to obtain a white solid with a yield of 80%, a liquid phase purity of 99.7%, and diastereoisomer of 0.14%.

### Example 18

10 g of crude product was taken and 100 ml of isopropanol was added, the mixture was heated to reflux at 90° C., 4.2 g of oxalic acid and 0.05 g of ethylamine were added, and insulation stirred for 30 min, then cooled in an ice water bath to 5° C. for crystallization, which was filtered, the filter cake was dried to obtain a white solid with a yield of 80%, a liquid phase purity of 99.8%, and diastereoisomer of 0.15%.

### Example 19

10 g of crude product was taken and 100 ml of isopropanol was added, the mixture was heated to reflux at 90° C., 4.2 g of oxalic acid and 0.05 g of ethanediamine were added, and insulation stirred for 30 min, then cooled in an ice water bath to 5° C. for crystallization, which was filtered, the filter cake was dried to obtain a white solid with a yield of 80%, a liquid phase purity of 99.8%, and diastereoisomer of 0.13%.

### Example 20

10 g of crude product was taken and 100 ml of isopropanol was added, the mixture was heated to reflux at 90° C., 4.2 g of oxalic acid and 0.05 g of triethylamine were added, and insulation stirred for 30 min, then cooled in an ice water bath to 5° C. for crystallization, which was filtered, the filter cake was dried to obtain a white solid with a yield of 80%, a liquid phase purity of 99.8%, and diastereoisomer of 0.14%.

### Example 21

10 g of crude product was taken and 100 ml of isopropanol was added, the mixture was heated to reflux at 90° C., 4.2 g of oxalic acid and 0.05 g of monoethanolamine were added, and insulation stirred for 30 min, then cooled in an ice water bath to 5° C. for crystallization, which was filtered, the filter cake was dried to obtain a white solid with a yield of 80%, a liquid phase purity of 99.6%, and diastereoisomer of 0.12%.

### Example 22

10 g of crude product was taken and 100 ml of isopropanol was added, the mixture was heated to reflux at 90° C., 4.2 g of oxalic acid and 0.05 g of dimethylformamide were added, and insulation stirred for 30 min, then cooled in an ice water bath to 5° C. for crystallization, which was filtered, the filter cake was dried to obtain a white solid with a yield of 80%, a liquid phase purity of 99.6%, and diastereoisomer of 0.15%.

### Example 23

10 g of crude product was taken and 100 ml of isopropanol was added, the mixture was heated to reflux at 90° C., 4.2 g of oxalic acid and 0.01 g of benzylamine were added, and insulation stirred for 30 min, then cooled in an ice water bath to 5° C. for crystallization, which was filtered, the filter cake was dried to obtain a white solid with a yield of 86%, a liquid phase purity of 99.9%, and diastereoisomer of 0.03%.

### Example 24

10 g of crude product was taken and 100 ml of isopropanol was added, the mixture was heated to reflux at 90° C. 4.2 g of oxalic acid and 0.1 g of benzylamine were added, and insulation stirred for 30 min, then cooled in an ice water bath to 5° C. for crystallization, which was filtered, the filter cake was dried to obtain a white solid with a yield of 84%, a liquid phase purity of 99.9%, and diastereoisomer of 0.02%.

### Example 25

10 g of crude product was taken and 100 ml of isopropanol was added, the mixture was heated to reflux at 90° C. 4.2 g of oxalic acid and 0.001 g of benzylamine were added, and insulation stirred for 30 min, then cooled in an ice water bath to 5° C. for crystallization, which was filtered, the filter cake was dried to obtain a white solid with a yield of 85%, a liquid phase purity of 99.9%, and diastereoisomer of 0.11%.

### Example 26

10 g of crude product was taken and 100 ml of isopropanol was added, the mixture was heated to reflux at 90° C., 4.2 g of oxalic acid and 1 g of benzylamine were added, and insulation stirred for 30 min, then cooled in an ice water bath to 5° C. for crystallization, which was filtered, the filter cake was dried to obtain a white solid with a yield of 65%, a liquid phase purity of 99.6%, and no diastereoisomer was detected.

### Example 27

10 g of crude product was taken and 100 ml of isopropanol was added, the mixture was heated to reflux at 90° C., 4.2 g of oxalic acid and 0.05 g of benzylamine were added, and insulation stirred for 30 min, then cooled to 0° C. for crystallization, which was filtered, the filter cake was dried to obtain a white solid with a yield of 86%, a liquid phase purity of 99.9%, and diastereoisomer of 0.01%.

### Example 28

10 g of crude product was taken and 100 ml of isopropanol was added, the mixture was heated to reflux at 90° C., 4.2 g of oxalic acid and 0.05 g of benzylamine were added, and insulation stirred for 30 min, then cooled to 10° C. for crystallization, which was filtered, the filter cake was dried to obtain a white solid with a yield of 84%, a liquid phase purity of 99.9%, and no diastereoisomer was detected.

### Example 29

10 g of crude product was taken and 100 ml of isopropanol was added, the mixture was heated to reflux at 90° C. 4.2 g of oxalic acid and 0.05 g of benzylamine were added, and insulation stirred for 30 min, then cooled to 30° C. for crystallization, which was filtered, the filter cake was dried to obtain a white solid with a yield of 81%, a liquid phase purity of 99.9%, and no diastereoisomer was detected.

### Example 30

10 g of crude product was taken and 100 ml of isopropanol was added, the mixture was heated to reflux at 90° C., 4.2 g of oxalic acid and 0.05 g of benzylamine were added, and insulation stirred for 30 min, then cooled to −20° C. for crystallization, which was filtered, the filter cake was dried to obtain a white solid with a yield of 88%, a liquid phase purity of 99.5%, and diastereoisomer of 0.11%.

### Comparative Example 1

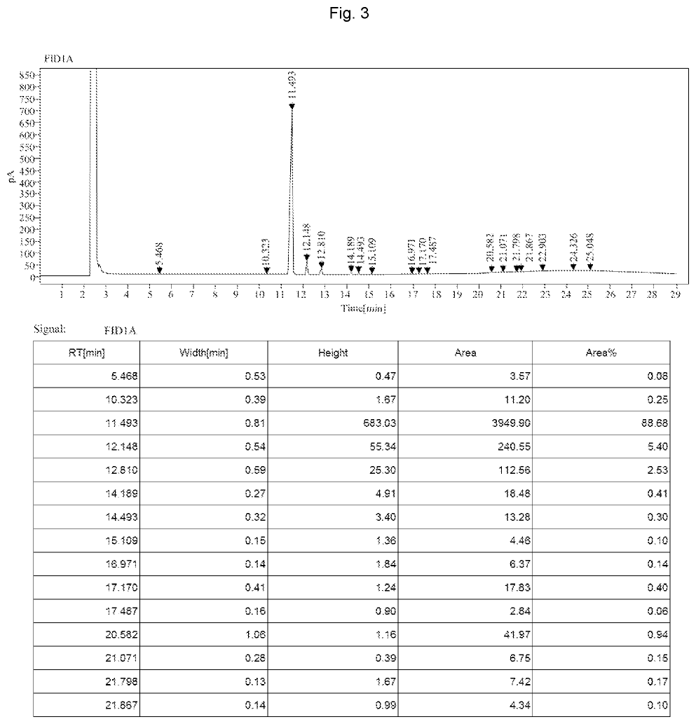

10 g of crude product was taken and 100 ml of isopropanol was added, the mixture was heated to reflux After dissolution, 4.2 g of oxalic acid in isopropanol (50 ml) was added dropwise. After the completion of dropwise addition, the temperature was maintained for 1 h, then cooled in an ice water bath, which was separated to obtain a waxy solid with a yield of 81%, a liquid phase purity of 88.7%, and diastereoisomer of 5.4%. The liquid phase chromatogram of the related substances in the product is shown in FIG. 3.

### Comparative Example 2

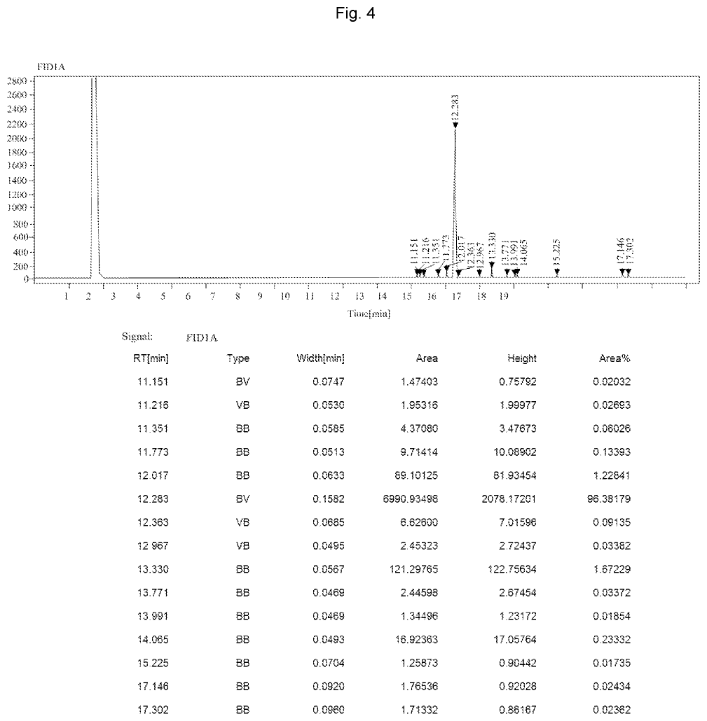

10 g of crude product was taken and 100 ml of isopropanol was added, the mixture was heated to reflux. After dissolution, 4.2 g of oxalic acid in isopropanol (50 ml) was added dropwise. After the completion of dropwise addition, the temperature was maintained for 1 h, then cooled at a constant rate of 5° C./h until it was cooled to 5° C. and maintained at 5° C. for 1 h, which was filtered to obtain a white solid with a yield of 75%, a liquid phase purity of 96.4%, and diastereoisomer of 1.7%. The liquid phase chromatogram of the related substances in the product is shown in FIG. 4.

As can be seen from the above, compared with the comparative examples, in the examples of the present disclosure, a new purification method for 3-amino-5-methylpiperidine with amino protected by Boc is used, which improves the product characters of 3-amino-5-methylpiperidine with amino protected by Boc after salifying and crystallization, thereby improving the yield and purity of the product, the purity of 3-amino-5-methylpiperidine with amino protected by Boc in the product is ≥99.5%, and the content of diastereoisomer impurities is ≤0.15%, at the same time, it simplifies the operation steps of salifying and crystallization of 3-amino-5-methylpiperidine with amino protected by Boc, shortens the operation time, and reduces production costs. In addition, it can be seen that when the purification parameters are within the preferred ranges of the present disclosure, the yield and purity of the product are better, and the detectable amount of diastereoisomer impurities is lower.

The descriptions above are merely preferred examples of the present disclosure and are not intended to limit the present disclosure, and various modifications and alterations of the present disclosure may be made by persons skilled in the art. Any modifications, equivalent substitutions, improvements and the like made within the spirit and principles of the present disclosure shall be included in the protection scope of the present disclosure.

What is claimed is:

1. A purification method for 3-amino-5-methylpiperidine with amino protected by Boc as shown in formula I, wherein comprises the following steps: 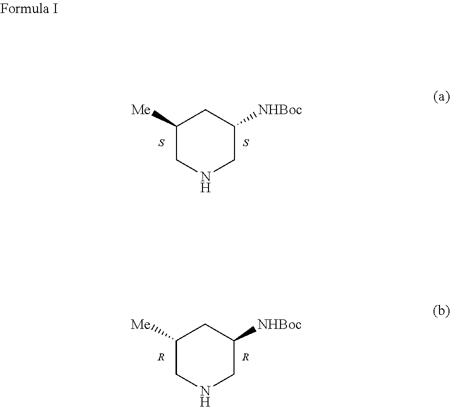
step S1, adding a solvent to the crude product of 3-amino-5-methylpiperidine with amino protected by Boc, heating until it is dissolved to obtain a mixed solution, the crude product comprises (3S,5 S) configuration product as shown in formula I (a) and/or (3R,5R) configuration product as shown in formula I (b);
step S2, adding an acid and an amine crystallization auxiliary to the mixed solution, stirring and dispersing to obtain a dispersed solution; and
step S3, cooling the dispersed solution until crystallization, separating and drying same to obtain a salt crystal of 3-amino-5-methylpiperidine with amino protected by Boc.

2. The purification method according to claim 1, wherein in the step S1, the solvent is one or more of methanol, ethanol, n-propanol, and isopropanol.

3. The purification method according to claim 1, wherein in the step S1, the liquid-solid ratio of the solvent to the crude product of 3-amino-5-methylpiperidine with amino protected by Boc is (4-16): 1.

4. The purification method according to claim 1, wherein in the step S1, the heating temperature is 40° C. to 100° C.

5. The purification method according to claim 1, wherein in the step S2, the acid is one or more of formic acid, acetic acid, methanesulfonic acid, p-toluenesulfonic acid, oxalic acid, succinic acid, lactic acid, and tartaric acid.

6. The purification method according to claim 1, wherein in the step S2, 0.001 mol to 0.01 mol of the acid is added to each gram of the crude product of 3-amino-5-methylpiperidine with amino protected by Boc in the mixed solution.

7. The purification method according to claim 1, wherein in the step S2, the amine crystallization auxiliary is one or more of ammonia, ethylamine, ethylenediamine, triethylamine, benzylamine, aniline, monoethanolamine, and dimethylformamide.

8. The purification method according to claim 1, wherein in the step S2, the mass ratio of the amine crystallization auxiliary to the crude product of 3-amino-5-methylpiperidine with amino protected by Boc in the mixed solution is (0.0001-0.1): 1.

9. The purification method according to claim 1, wherein in the step S2, the dispersion temperature is 40° C. to 100° C.

10. The purification method according to claim 1, wherein in the step S3, the crystallization temperature is −20° C. to 30° C.

11. The purification method according to claim 1, wherein in the step S2, the acid is one or more of oxalic acid, lactic acid, and tartaric acid.

12. The purification method according to claim 1, wherein in the step S2, the amine crystallization auxiliary is benzylamine and/or aniline.

13. The purification method according to claim 1, wherein in the step S2, the mass ratio of the amine crystallization auxiliary to the crude product of 3-amino-5-methylpiperidine with amino protected by Boc in the mixed solution is (0.001-0.01): 1.

14. The purification method according to claim 1, wherein in the step S3, the crystallization temperature is 0° C. to 10′C.

15. The purification method according to claim 2, wherein in the step S1, the heating temperature is 40° C. to 100° C.

16. The purification method according to claim 2, wherein in the step S2, the acid is one or more of formic acid, acetic acid, methanesulfonic acid, p-toluenesulfonic acid, oxalic acid, succinic acid, lactic acid, and tartaric acid.

17. The purification method according to claim 2, wherein in the step S2, 0.001 mol to 0.01 mol of the acid is added to each gram of the crude product of 3-amino-5-methylpiperidine with amino protected by Boc in the mixed solution.

18. The purification method according to claim 2, wherein in the step S2, the amine crystallization auxiliary is one or more of ammonia, ethylamine, ethylenediamine, triethylamine, benzylamine, aniline, monoethanolamine, and dimethylformamide.

19. The purification method according to claim 2, wherein in the step S2, the mass ratio of the amine crystallization auxiliary to the crude product of 3-amino-5-methylpiperidine with amino protected by Boc in the mixed solution is (0.0001-0.1): 1.

20. The purification method according to claim 2, wherein in the step S2, the dispersion temperature is 40° C. to 100° C.
